(19) United States
(12) Patent Application Publication
(10) Pub. No.: US 2025/0179466 A1
(43) Pub. Date: Jun. 5, 2025

(54) CHROMATOGRAPHIC PURIFICATION METHOD AND USES THEREOF

(71) Applicant: ChromaCon AG, Zürich (CH)

(72) Inventors: Lars AUMANN, Zürich (CH); Richard WELDON, Zürich (CH); Sebastian VOGG, Basel (CH); Thomas MÜLLER-SPÄTH, Zürich (CH)

(73) Assignee: ChromaCon AG, Zürich (CH)

(21) Appl. No.: 18/842,967

(22) PCT Filed: Feb. 28, 2023
(86) PCT No.: PCT/EP2023/054895

§ 371 Date: Aug. 30, 2024

(30) Foreign Application Priority Data
Mar. 3, 2022 (EP) 22159938.4

(51) Int. Cl. C12N 15/10; B01D 15/10; B01D 15/16; B01D 15/18; B01D 15/20; B01D 15/24

(52) U.S. Cl. CPC C12N 15/101; B01D 15/102; B01D 15/166; B01D 15/1871; B01D 15/203; B01D 15/245

(57) ABSTRACT

A chromatographic purification method for the isolation of a product from a feed mixture using two columns, wherein in one step b) the first upstream column is loaded with feed, followed by at least one interconnected step c), wherein in the interconnected step c) the first column is fed with eluent with a gradient, wherein the stream exiting the first column is adjusted inline with inline adjustment eluent before entering the second column during the period of gradient elution, and wherein the inline adjustment eluent is as the eluent fed at the first column inlet but controlled to have a higher or lower modifier concentration than the eluent exiting at the first column, and wherein the modifier difference of the inline adjustment eluent is chosen such that the adherence of the product to the stationary phase of the second column is higher than without that difference.

### TECHNICAL FIELD

The present invention relates to a chromatographic purification method as well as to uses thereof.

### PRIOR ART

Most production processes of therapeutic substances such as therapeutic peptides, oligonucleotides and proteins include several chromatographic purification steps.

Thereby, each step contributes to the achievement of an overall purity specification for the target compound, achieved through removal of process- and product-related impurities from the starting material.

From the perspective of manufacturing costs during purification process development it is desirable to minimize the number of chromatographic steps, while ensuring that final purity specifications are met. In addition, for process economics, it is desired to maintain a high yield of the target compound and a high throughput.

Linear gradient chromatography is an important mode of operation to achieve a good compromise between purity and throughput, especially for complex biomolecules such as proteins, peptides, and oligonucleotides. However, even when operated in linear gradient mode, a trade-off between purity and yield/throughput is inherent to traditional single column processes, so called batch processes. The trade-off is mainly caused by product-related impurities that overlap with the product in the chromatograms observed in the purification processes. When eluting from the column, the overlaps lead to only partially pure side-fractions that may not be included in the product pool as they do not meet purity specifications. These side fractions can be collected and recycled for product recovery, representing a considerable extra effort for handling, storage and analysis. Alternatively, they are discarded, representing a waste of product and effort that was made to generate the starting material and a reduction of yield.

In short, in case of discard of these side fractions, part of the product is lost, while in case of recycling of these side fractions, the overall productivity (throughput) of the process suffers.

In theory, the overlaps can at least partly be resolved by running shallower linear gradients and extending the chromatographic run, but again, this leads to reduced productivity (throughput).

Several recycling processes using a single column have been developed for automatic recycling of impure side-fractions. However, most of these processes are not suitable for operation in linear gradient mode (constrained to isocratic operation) as they are run in a closed loop. Often, the chromatographic profiles are passed through the pumps (see e.g. so called-steady state recycling processes, SSR, C. M. Grill, L. Miller/J. Chromatogr. A 827 (1998) 359-371). A process using 2 columns, where the profile does not pass through the pumps is suggested in U.S. Pat. No. 5,630,943. However, also this process does not allow operation in linear gradient mode.

Likewise, 4-zone Simulated Moving Bed (SMB) processes are not capable of linear gradient mode and ternary purification can only be achieved by complex SMB setup, such as two SMB processes in series (see e.g. J. S. Hur, P. C. Wankat, Ind. Eng. Chem. Res. 2006, 45, 1426-1433)

A few recycling processes, capable of linear gradient mode using multicolumn chromatographic processes have been presented, such as MCSGP disclosed in EP-A-1 877 769, GSSR (Silva et al, J Chromatogr A 2010 Dec. 24; 1217 (52): 8257-69), and a process according to EP 21 199 535.2. These processes use recycling of partially pure side fractions from a first column and re-adsorption on a second column. Re-adsorption on the second column can be ensured by inline adjustment of the side fractions after they have left the first column, and before they enter the second column. After side-fraction recycling in interconnected mode, the columns are disconnected for loading of fresh feed, for product elution and for column cleaning. These processes work well, yet there is quite some effort required to find suitable operating points.

MCSGP has been described for 2-8 column configurations but in industry today the 2-column configuration is used due to lower equipment complexity and higher operational flexibility.

US-A-2013248451 proposes a chromatography system for separation of a biopolymer, comprising at least one feed tank, at least one hold tank, at least one elution buffer tank, at least one eluate tank, at least two packed bed chromatography columns and for each packed bed chromatography column at least one pump and at least one outlet detector both connected to said each packed bed chromatography column, wherein the feed tank, the hold tank(s), the elution buffer tank and the eluate tank are each connected to the packed bed chromatography columns via a system of valves.

De Luca et al. in: “Process Intensification for the Purification of Peptidomimetics: The Case of Icatibant through Multicolumn Countercurrent Solvent Gradient Purification (MCSGP)”, INDUSTRIAL & ENGINEERING CHEMISTRY RESEARCH, vol. 60, no. 18, report that biopharmaceuticals are subjected to very strict purity requirements to be marketed. At the same time, peptides and other biomolecules are industrially synthesized through techniques (e.g., solid-phase synthesis) often leading to the formation of many impurities with molecular characteristics very similar to the target product. Therefore, the purification of these mixtures via preparative chromatography can be very challenging. This typically involves ternary or central-cut separations, characterized by chromatograms where the central peak, corresponding to the target product, exhibits significant overlapping on both sides with impurities slightly more or less adsorbable. In single-column (batch) preparative chromatography, this leads to a typical yield-purity tradeoff, meaning that high purity can be obtained at the cost of low yield and vice versa, with obvious consequences on the overall production costs. The study demonstrates how this limitation can be alleviated using the continuous countercurrent operating mode, conducted on a multicolumn system, as a tool for process intensification. In particular, the Multicolumn Countercurrent Solvent Gradient Purification (MCSGP) process has been applied to the purification of an industrial crude mixture of icatibant, which is a peptidomimetic antagonist of bradykinin B2-receptor that has been recently also considered for the treatment of patients affected by COVID-19 disease. It is shown that MCSGP allows conjugating process simplicity (using only two columns) with a significant improvement in process performance, compared to the corresponding batch process. This includes all process performance parameters: yield, productivity, and buffer consumption for a given purity specification of icatibant.

Steinebach et al. in “Experimental design of a twin-column countercurrent gradient purification process”, JOURNAL OF CHROMATOGRAPHY A, ELSEVIER, AMSTERDAM, NL, vol. 1492, report, that as typical for separation processes, single unit batch chromatography exhibits a trade-off between purity and yield. The twin-column MCSGP (multi-column countercurrent solvent gradient purification) process allows alleviating such trade-offs, particularly in the case of difficult separations. In the presented work an efficient and reliable procedure for the design of the twin-column MCSGP process is developed. This is based on a single batch chromatogram, which is selected as the design chromatogram. The derived MCSGP operation is not intended to provide optimal performance, but it provides the target product in the selected fraction of the batch chromatogram, but with higher yield. The design procedure is illustrated for the isolation of the main charge isoform of a monoclonal antibody from Protein A eluate with ion-exchange chromatography. The main charge isoform was obtained at a purity and yield larger than 90%. At the same time process related impurities such as HCP and leached Protein A as well as aggregates were at least equally well removed. Additionally, the impact of several design parameters on the process performance in terms of purity, yield, productivity and buffer consumption is discussed. The obtained results can be used for further fine-tuning of the process parameters so as to improve its performance.

EP 2 682 168 discloses processes and systems for the purification of biological molecules including therapeutic antibodies and Fc-containing proteins. The process for the purification of a biological target molecule comprises the steps of: a) providing a sample comprising the target molecule and one or more impurities, b) removing one or more impurities by centrifugation and/or filtration and/or settling; c) subjecting the liquid phase resulting from step (b) to a bind and elute chromatography step whereby at least two separation units are used and whereby all separation units have the same matrix; d) contacting the liquid comprising the target molecule resulting from step (c) with two or more matrices in a flow-through mode, whereby one of the matrices is an anion exchange matrix, whereby a single bind and elute chromatography step is present.

US2014248643 discloses a chromatographic process for the enrichment of at least one compound of interest from a mixture, using chromatographic columns, wherein said process involves a sequence of the following steps: (i) a cyclic accumulation phase, in which the chromatographic columns are alternatingly operated in an interconnected phase, followed by a disconnected phase, wherein subsequently columns exchange places and wherein the phases are carried out sequentially; (ii) a cyclic separation phase, in which the chromatographic columns are alternatingly operated in an interconnected phase, followed by a disconnected phase, wherein after these phases columns exchange places to undergo the next interconnected and disconnected phases; and (iii) an elution phase, in which from the column, which at the end of phase (i) or at the end of phase (ii) contains the compound of interest, is extracted via the outlet.

### SUMMARY OF THE INVENTION

The present invention aims at providing an improved mode of operation of linear gradient chromatography, allowing higher purity than conventional batch chromatography.

It was surprisingly found that through introduction of an inline adjustment flow in between a setup of two interconnected columns of preferably the same type, the separation of compounds can be improved during gradient operation, preferably linear gradient operation, while the product-containing chromatographic profile moves from the first column to the second column. The resulting product pool has a higher purity than a product pool collected from a setup corresponding to standard batch chromatography, i.e. a product pool from a run with a (linear) gradient through two columns in series without inline adjustment. The inline adjustment flow rate in the suggested process is thereby selected such that it improves adsorption of the compounds to be separated by lowering the modifier concentration, yet it does not lead to full adsorption of the compounds to be separated, allowing them to elute in reasonable time such that a good process throughput is warranted. The (linear) gradient comprises at least one (linear) gradient segment, i.e., e.g. multilinear gradients including several gradient segments with different slopes are possible.

In the presented process, the first column performs a chromatographic procedure including the tasks of regular (linear) gradient operation, i.e., the column is equilibrated, loaded with feed, washed, eluted by a (linear) gradient, and regenerated. However, in contrast to regular linear batch chromatography, the outlet stream containing the product P and leaving the outlet of the first column is not directly collected, fractionated, or sent to waste, but it is directed to a second column, and before the stream enters the second column, it is inline adjusted, e.g. by means of a pump during the period of (linear) gradient elution. At this stage, the second column is in an equilibrated state. Before entering the second column, the stream may be mixed upon or after inline adjustment, however the stream is not kept in a hold tank (which inter alia provides for a clear distinction from US-A-20130248451), which would destroy the partial separation achieved in the first column and prevent use of displacement effects. After the second column, the product is collected, and waste is discarded, however there may also follow yet another further column, again with yet another inline adjustment, repeating the function of the second column. Collection of the product may be triggered through a detector signal. In the presented process, the load of feed always occurs on the same column, the upstream column 1, never on the downstream column 2.

The different phases of a process according to the invention are schematically illustrated in FIG. 1, which shows two different embodiments of such a process (mode 1 and mode 2). Mode 1 (left) shows the process with permanent connection of column 1 and column 2, while mode 2 (right) shows the process with interconnection of columns 1 and 2 only when operationally necessary and operation in single column mode whenever possible. One reason for single column operation can be the reduced backpressure due to operation with half column bed height, and the possibility to run a shorter residence time. This allows completion of the run in a shorter time period, hence throughput/productivity can be improved.

In both modes, the process as illustrated comprises or consists of the typical steps of linear gradient chromatography:
a.) Equilibration, typically carried out by subjecting the column(s) to the solvent in constant composition used for the subsequent purification process, wherein the solvent is in a composition preferably identical to the one at the beginning of the purification process and/or to the ones of the feed; this equilibration can be done in interconnected mode or it can be done with each of the columns individually;
b.) Feed and wash, this is normally done sequentially, after the feeding of column 1 in a first step isocratic gradient elution takes place; this step can be carried out either in interconnected mode, or it can be carried out just with the upstream column 1, while column 2 is either resting or continued to be equilibrated;
c.) Linear gradient elution, during this phase mandatorily the 2 columns are interconnected, i.e. the outlet of the upstream column 1 is connected to the inlet of the downstream column 2; this step includes the inline adjustment according to the invention;
d.) Regeneration; this step normally involves subjecting the columns either individually or in interconnected mode to at least one solvent for cleaning.

In both modes, during step c.) linear gradient elution, the product-containing fractions are transferred from column 1 to column 2 in interconnected mode, subject to inline adjustment in-between the columns. After the downstream column 2 the stream is eluted and fractionated/collected.

More precisely, during a.) equilibration, in mode 1, the columns are interconnected and equilibrated in series, while in mode 2, the columns are disconnected and equilibrated separately.

During step b.) feed and wash, in mode 1, the columns are interconnected. Inline adjustment during this step is possible if it is expected that product P is transferred to column 2 during feed or wash, which is however usually not the case if appropriate feed solvent and mobile phases are selected. In mode 2 in step b.), during feed and wash, the feed is loaded onto column 1 and the column 1 subsequently is washed while the outlet of column 1 is directed to waste. In this step, first weakly adsorbing impurities W, but no product P, may be eluted from column 1. In this step, in mode 2, column 2 can be inactive or it may be continued to be equilibrated.

The next step, c.) linear gradient elution, is identical in modes 1 and 2: A linear gradient is operated on column 1 interconnected with column 2, and the product P, together with overlapping impurities W and S is transferred from column 1 to column 2 with inline adjustment in between the two columns. The chromatographic profile leaving column 2 is collected as pool or fractionated to recover the product P.

In the next step, d.) regeneration, in mode 1, the columns are interconnected, and regenerated in series, while in mode 2, the columns are disconnected and regenerated separately.

The inline adjustment is performed with water, or a solvent or buffer with lower modifier content such that the inline-adjusted stream corresponds to a linear gradient over time with lower starting and end concentrations. It can be shown by process simulations using a mechanistic model that the lowering of the gradient concentration by inline adjustment can lead to a sharpening of the peak profiles and enhanced displacement effects of the compounds to be separated. Therefore, the process works best for compounds that show displacement effects under the selected chromatographic conditions in the first place. In thermodynamic terms, the process is dependent on the adsorption equilibrium (isotherm) of the compounds to be separated and works best in case of adsorption behavior that can be described by Langmuir-type competitive isotherms and derivatives thereof. Using the suggested process for exploiting the displacement effects enables higher purity values that are inaccessible to standard chromatography. Therefore, the number of chromatography steps required for the purification can be possibly reduced.

The inline adjustment flow rate preferably is in the same order of magnitude as the flow rate entering the first column. No inline adjustment is required in process steps that do not include transfer of product from column 1 into column 2.

FIG. 2 shows the process setup with the various options for mixing after inline adjustment. There can be a dynamic mixer (Option A), a static mixer (Option B), a piece of capillary/tubing/piping (Option C), or no mixer (Option D).

Note that the mixer volume should be optimized to ensure proper mixing for establishing the “adjusted” gradient while it should not cancel the partial separation of the compounds to be separated that leave the first column. It is possible to perform inline adjustment and mixing in the same device, i.e., saving the T-Piece.

In a preferred embodiment, Option A, the mixer is a dynamic mixer, for example a chamber with a magnetic stir bar that is actuated. As mentioned, it is possible to perform mixing and inline adjustment in the same mixer device as typical dynamic mixing devices are used for gradient mixing and therefore have two inlets and one outlet which can be used in the setup according to FIG. 2.

In a preferred embodiment, Option B, the mixer is a static mixer, i.e., a mixing chamber with baffles or other means to induce back-mixing.

In a preferred embodiment, Option C, the mixer is a piece of capillary/tubing/piping (depending on the scale of the equipment), with a different diameter than the capillary/tubing/piping used at the outlets of column 1 and inlet of column 2. Depending on the flow rate, a turbulent flow is induced, leading to cross-mixing in the mixing piece. The turbulence of the flow can be estimated by the Reynolds number which increases with decreasing capillary/tubing/piping diameter. Reynolds numbers above 2300-3000 are expected to lead to turbulent flow.

Additional back-mixing occurs through the change of diameter (constriction and expansion) at the start/end of the capillary/tubing/piping piece. The Reynolds number increases with increasing flow rate.

In a preferred embodiment, Option D, there is no mixing device. In some cases, sufficient mixing is provided readily through a tubing/piping connection of the same diameter as the regular tubing/piping connection, i.e. through a direct connection of point of inline adjustment (T-Piece) and the inlet of column 2. This can be the case at high linear flow rates and mobile phases with a low viscosity.

In a preferred embodiment, apart from the detector at the outlet of the second column, that is used to monitor the chromatogram and possibly to initiate product collection, a further detector is positioned at the outlet of column 1 before the point of inline adjustment. The signal monitored by this detector can be used to gather information on the chromatographic profile, for example for online changes of the chromatographic methods, such as determination of the end-point of elution and start of the regeneration or for observing the resolution of column 1 to detect column degradation. This setup is shown in FIG. 3.

In a preferred embodiment, the columns of the suggested process are disconnected for faster equilibration and regeneration steps. For example, when disconnected, pump P1 can deliver fluid to column 1, while pump P2 can deliver fluid to column 2, and the outlets of the columns can be directed to waste. In practice, this mode of operation can be realized by valve switching in suitable chromatographic equipment. This mode of operation can be also useful for testing the two columns independently to determine if exchange of one of the columns, or both, is necessary.

In a preferred embodiment the two columns, the point of inline adjustment, and the mixer are integrated in a single separation device such that they form compartments of a larger column with suited spatial separation and a liquid inlet in between the two compartments, such that the dead volume between the columns is minimized and the separation device can be connected to the chromatography system much faster.

In a preferred embodiment, the two columns are monoliths or membranes are used instead of the columns.

In another preferred embodiment, the two columns contain different stationary phases.

In a preferred embodiment, it is possible to run the suggested process with a minimal number of pumps, i.e. with just one gradient pump and to divert the stream for inline adjustment from the gradient pump (channel with low modifier concentration) using an appropriate piece of hardware such as a diverter valve to split the stream and flow meters for pump and valve control. The same pump may also deliver the feed through a multi-position selection valve. In other words, the diverter valve substitutes the inline adjustment pump and the multi-position valve substitutes feed pump. Obviously, it is possible to use just one of the two substitutions. The setup with both substitutions is shown in FIG. 4.

In a further preferred embodiment, the setup according to FIG. 4 is equipped with a detector after column 1 as described above.

In a preferred embodiment, the columns of the suggested process have different bed volumes while containing the same stationary phase. Different bed volumes can be used for optimizing residence time, back-pressure and process performance, while using the same stationary phase ensures that the process can be regarded as a single unit operation from a regulatory standpoint.

In the presented process it may happen that column 1 ages at a faster rate than column 2, as it is loaded with crude feed during operation, while column 2 only receives fluids that have been pre-purified by column 1 and/or fluids that are freshly prepared (inline-adjustment buffer/solvent, equilibration buffer/solvent, regeneration buffer/solvent). If needed, the presented process allows for exchange of the two columns independently for purposes of re-packing or replacement, but only after the product has been completely eluted (exchange) or the columns have been regenerated (re-packing), i.e. there is no exchange of the positions of the two columns in any of the phases where the mixture to be separated is in the system.

More generally speaking, the present invention proposes a chromatographic purification method for the isolation of a product P from a feed mixture F consisting of the desired product P and at least two further components, a component with impurities W more weakly adsorbing than the desired product and a component with impurities S more strongly adsorbing than the desired product. According to the invention therefore the feed mixture is a ternary mixture comprising the product as well as impurities on both sides of the product considering the chromatogram.

The proposed method uses two or more chromatographic columns, preferably however only two columns are used.

A first upstream column has a first column inlet and a first column outlet, and a second downstream column has a second column inlet and a second column outlet.

The method includes at least one step b), in which the first upstream column is loaded with feed (F) via the first column inlet, the feed step can be followed by a washing step.

This loading step is followed by at least one interconnected step c) in which the columns are interconnected in series for passing eluate containing product P via the first column outlet to the second column inlet.

According to the invention, in said at least one interconnected step c) the first column is fed with eluent with a gradient in the form of a temporally changing modifier concentration.

Furthermore, in said at least one interconnected step c) the stream exiting the first column outlet is adjusted inline with inline adjustment eluent before entering the second column inlet at least during the period of gradient elution.

Even further and importantly, the inline adjustment eluent is as the eluent fed at the first column inlet (which means it preferably comprises or consists of the same base solvent and the same modifier) but controlled to have a higher or lower modifier concentration than the eluent exiting at the first column outlet. A particularly simple and preferred implementation of the inline adjustment is possible if the adjustment eluent is just given by essentially pure base solvent (in particular for an increasing modifier concentration at the inlet of the upstream column for the gradient) or by pure modifier solvent (in particular for a decreasing modifier concentration at the inlet of the upstream column for the gradient).

In doing so, the modifier difference of the inline adjustment eluent is chosen such that the adsorption of the product on the stationary phase of the second column is stronger than without that difference, promoting aforementioned displacement effects and improving the separation. Inline adjustment correspondingly leads to a better separation at the outlet of the downstream column without requiring more time to do so.

According to a preferred embodiment of the proposed method, in said at least one interconnected step c) the first column is fed with solvent with an at least segment wise linear gradient (also called multilinear gradient), preferably with a continuous linear gradient during the whole of said interconnected step c).

Preferably, the ratio of the flow rate of solvent with gradient in said at least one interconnected step c) at the first column inlet to the flow rate of the inline adjustment is between 5:1 and 1:5.

According to the invention, the method either involves only 2 columns, and in said at least one interconnected step c) product (P) is collected at the second column outlet.

However, the function of the second downstream column can also be repeated equivalently by further downstream columns. According to another variant of the invention, therefore the method involves said first and said second column and at least one further column, fulfilling downstream of said second column, the equivalent function of said second column in that the stream exiting the second column is adjusted in line with inline adjustment solvent before entering the further column inlet at least during the period of gradient elution, wherein the corresponding inline adjustment solvent is the same as the solvent fed at the first column inlet but controlled to have a different modifier concentration than the solvent exiting at the second column outlet, and wherein the modifier difference of the inline adjustment solvent is chosen such that the adherence of the product to the stationary phase of the further column is higher than without that difference, and wherein in said at least one interconnected step c) product P is collected at the further column outlet.

Preferably, a detector is positioned at the outlet of the second column that is used to monitor the chromatogram, and collection of the product P is performed at the outlet of the second column 2 controlled by said detector signal.

Also a detector can be positioned at the outlet of the first column upstream of the point of inline adjustment for controlling inline adjustment and/or product collection.

The detectors can be UV detectors, IR detectors, VIS detectors, Raman detectors, but also may entail means for measuring modifier concentration, i.e. pH and/or salt concentration, and the detectors may be combinations of such detection schemes.

Typically, in said at least one interconnected step c) during the transfer of eluate containing the product (P) from the first column outlet to the second column inlet the stream entering the second column (2) is mixed inline after or at the point of inline adjustment, by means of a dynamic mixer, a static mixer or a piece of piping with a different diameter than used at the first column outlet and the second column inlet.

The columns can be disconnected and operated as single columns during at least one part of the procedure that different from said at least one interconnected step c), in particular at least one of the steps of a) equilibration, b) feed and/or wash, d) regeneration.

Normally, the method includes or rather further preferably consists of the following steps:
a) equilibration of the columns, either in interconnected mode or each column separately, followed by
b) feed, either in interconnected mode or only with the first column disconnected from the second column, in that said feed mixture F is introduced via the first column inlet, preferably followed by washing by introducing solvent without feed mixture by the first column inlet, followed by
c) said at least one interconnected step, followed by
d) regeneration, either in interconnected mode or each column separately, by using conditions cleaning the columns,
wherein the sequence of steps a)-d) can be repeated as often as desired and necessary, preferably at least 10 times, or at least 100 times, and wherein further preferably between steps d) and a) in such repetition the position of the columns can be interchanged to make sure the usage of the upstream column is not higher than the one of the downstream column or vice versa, either after each sequence, or after each 5th or 10th repetition.

A diverter valve can be used to provide the stream required for inline adjustment in between the two columns.

The first column and the second column may have the same or different bed volumes.

The modifier is preferably selected from the group consisting of an organic or inorganic solvent or mixture thereof different from a base solvent or mixture thereof of the eluent, an electrolyte in such an organic or inorganic solvent (or a mixture thereof), preferably selected from a dissolved salt or a pH, or a combination thereof.

Said base solvent can be water or a mixture of water with at least one organic solvent or water in a mixture with one or more salts and/or organic solvents one or both in a minor proportion compared with water.

Said modifier can be an organic solvent or a mixture of water with at least one organic solvent having a higher concentration of said at least one organic solvent than in the base solvent, water or a mixture of water with at least one organic solvent with a different salt or H⁺ concentration than the base solvent.

The two columns, the point of inline adjustment and the mixer can be integrated in a single separation device such that they form compartments of a larger column with suited spatial separation and a liquid inlet in between the two compartments.

The two or more columns may also be structured as monoliths or membranes.

According to a further preferred embodiment, the inline adjustment is run as a flow-rate gradient.

In said at least one interconnected step c) the first upstream column is preferably fed with eluent consisting of a base solvent in mixture with a modifier with a gradient in the form of a temporally changing modifier concentration. The stream exiting the first column outlet can then preferably be adjusted in line with inline adjustment eluent before entering the second column inlet at least during a period of gradient elution, in that the adjustment eluent consists of the base solvent without modifier.

The first and second column preferably contain the same packing materials, however they may also contain different packing materials.

Furthermore, the present invention relates to the use of such a method for the purification of biomolecules, of natural or synthetic origin, preferably selected from the group consisting of nucleic acid molecules, including DNA and RNA molecules, peptides, proteins, including antibodies, carbohydrates, lipids as well as combinations and modifications as well as fragments thereof.

Further embodiments of the invention are laid down in the dependent claims.

### BRIEF DESCRIPTION OF THE DRAWINGS

Preferred embodiments of the invention are described in the following with reference to the drawings, which are for the purpose of illustrating the present preferred embodiments of the invention and not for the purpose of limiting the same. In the drawings,

FIG. 1 shows a schematic of two operating modes of the presented process; arrows indicate the direction of fluid flow;

FIG. 2 shows a schematic of the presented process including Options A-D for mixing devices, and a detector at the outlet of column 2;

FIG. 3 shows the schematic of the process in FIG. 1 with additionally a detector positioned between the outlet of column 1 and the point of inline adjustment;

FIG. 4 shows a schematic of the process in FIG. 1 with minimal number of pumps;

FIG. 5 shows the experimental purity-yield curve for the purification of ssRNA using the standard batch process and the presented process (example 1);

FIG. 6 shows the chromatograms at the outlet of column 2 obtained by means of the simulations for the purification of Angiotensin II using the standard batch process and the presented process (example 2);

FIG. 7 shows the simulated purity-yield curve for the purification of Angiotensin II using the standard batch process and the presented process (example 2);

FIG. 8 shows the simulated load-yield curve for the purification of Angiotensin II using the standard batch process and the presented process (example 2);

FIG. 9 shows the simulated yield-inline adjustment flow curve for the purification of Angiotensin II using the standard batch process and the presented process (example 3).

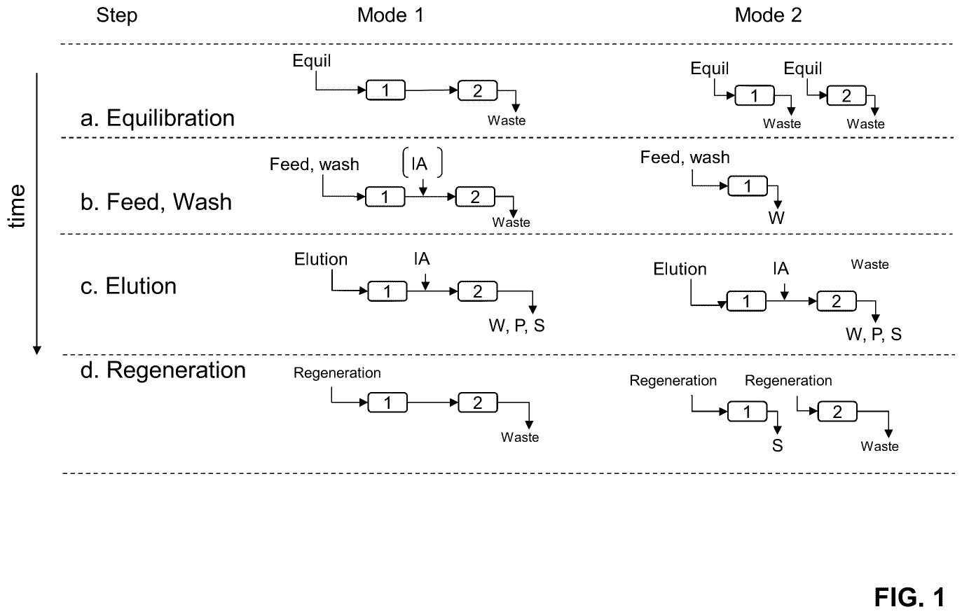

### DESCRIPTION OF PREFERRED EMBODIMENTS

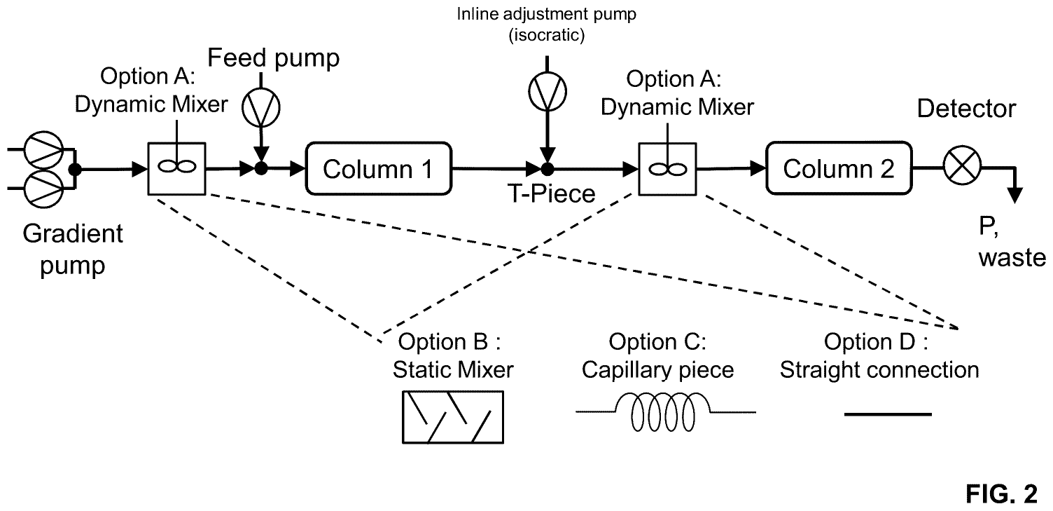

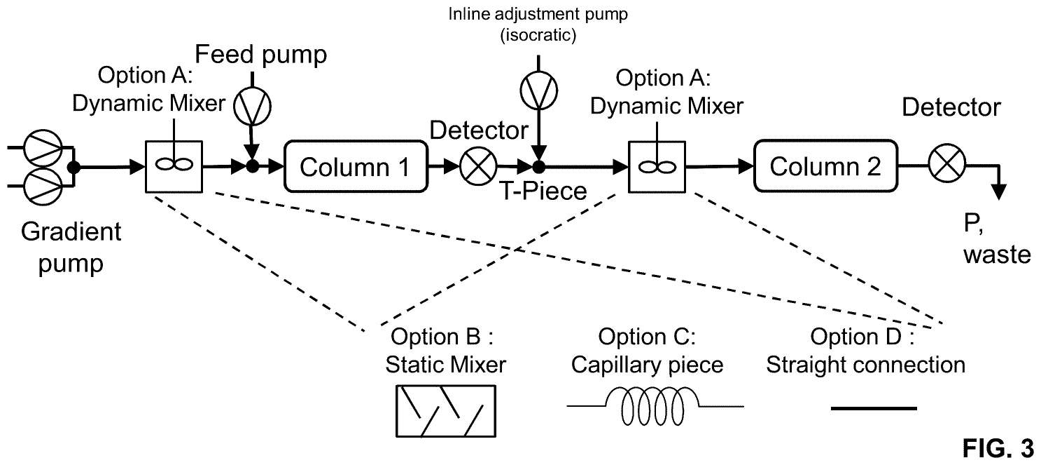

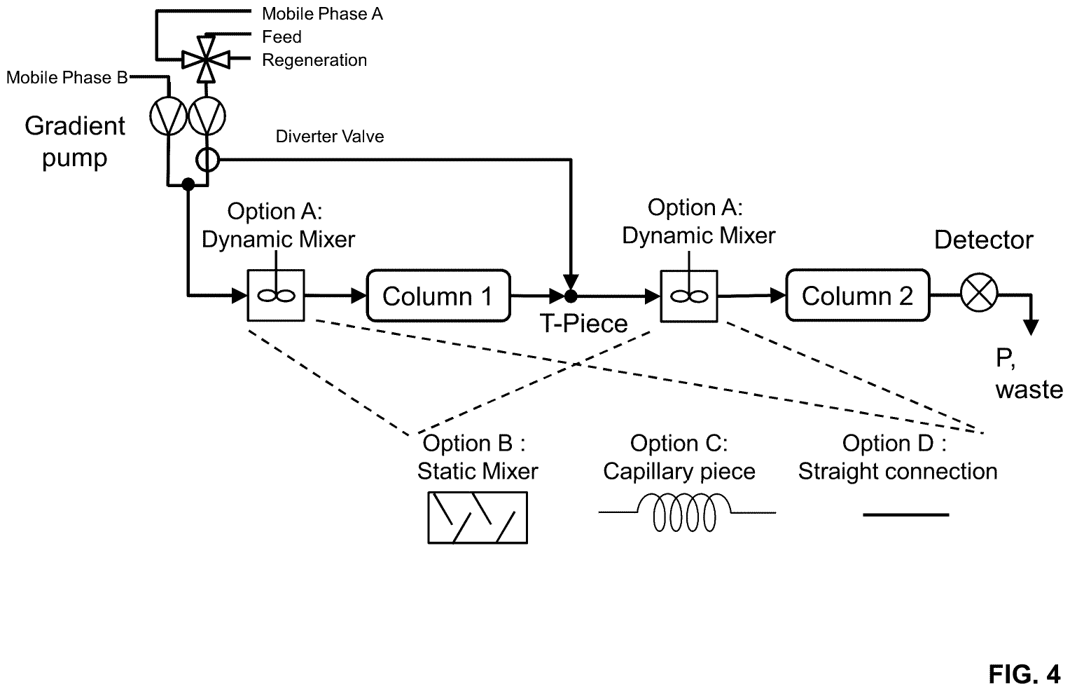

Example 1: Purification of RNA: The presented process setup of FIG. 2 was operated for the purification of a 20-mer single-stranded RNA (ssRNA) and compared to the same setup without inline adjustment. The following experimental conditions were used: The process was operated using a Contichrom CUBE 30 system from ChromaCon AG, Switzerland. Two columns packed with YMC Triart Prep C18S 150×4.6 mm 15 μm 120 A, were used. Mobile phase A was 99% 0.2 M Sodium Acetate, 1% Acetonitrile; mobile phase B was 99% 0.2 M Sodium Acetate, 10% Acetonitrile; the feed was dissolved in mobile phase A to a concentration of 1.0 g/L. The load in both cases was 10 g/L. The feed was supplied using the pump P3 of the Contichrom CUBE system, while the gradient was prepared using the gradient Pump P1, and in case of the presented process, inline adjustment was performed with Pump P2, running mobile phase A. Inline adjustment was active always except during the feed step. For further process optimization one could have used inline adjustment only during linear gradient elution. No mixing device was used at or after inline adjustment.

In the standard process, the flowrate of P2 was zero. The run duration was at the same in both cases (188 min).

An overview of the flow rate and gradient settings is provided in Table 1:

TABLE 1
Operating parameters of Example 1

| | | | Standard batch | | | Proposed process | | |
|---|---|---|---|---|---|---|---|---|
| | gradient | time | mL/min | mL/min | mL/min | mL/min | mL/min | mL/min |
| Procedure | % B | min | (P1) | (P2) | (P3) | (P1) | (P2) | (P3) |
| Equilibration | 10 | 7.5 | 1.0 | 0 | 0 | 1.0 | 0.2 | 0 |
| Feed | — | 25 | 0 | 0 | 2.0 | 0 | 0 | 2.0 |
| Wash | 15 | 10 | 1.0 | 0 | 0 | 1.0 | 0.2 | 0 |
| Gradient | 95 | 115 | 1.0 | 0 | 0 | 1.0 | 0.2 | 0 |
| Hold | 95 | 15 | 1.0 | 0 | 0 | 1.0 | 0.2 | 0 |
| Strip | 95 | 5 | 1.0 | 0 | 0 | 1.0 | 0.2 | 0 |
| Re-Equilib | 10 | 10 | 1.0 | 0 | 0 | 1.0 | 0.2 | 0 |

Fractionation of the effluent of column 2 was performed using the R1 fraction collector of the CUBE system at 2 mL fraction size. Fraction analysis was performed using an Agilent UHPLC 1200 system equipped with a YMC Triart C18 100×2 mm 1.9 μm 12 nm column; mobile phase A was hexafluorisopropanol 100 mM (HFIP)+4 mM triethylamine (TEA) in water and mobile phase B was methanol (MeOH).

For each analysis, the injection volume was 1 μL and the method applied a constant flow rate of 0.2 mL/min performing a first linear gradient from 5% B to 10% B in 2 minutes followed by a second linear gradient from 10% B to 20% B in 17 minutes. The column thermostat was set to 60° C. and the reference wavelength was 260 nm. The starting feed material had a purity value of 69.6%.

The results of the sample analysis were plotted as purity-yield curve. Starting from the fraction with highest purity as first data point, neighboring fractions were included one by one and a new purity and yield value was calculated based on the impurity content and product concentration of the combined pool, leading to the purity-yield curve. The procedure was carried out for the fraction analysis results of the regular batch process (without inline adjustment) and the presented process.

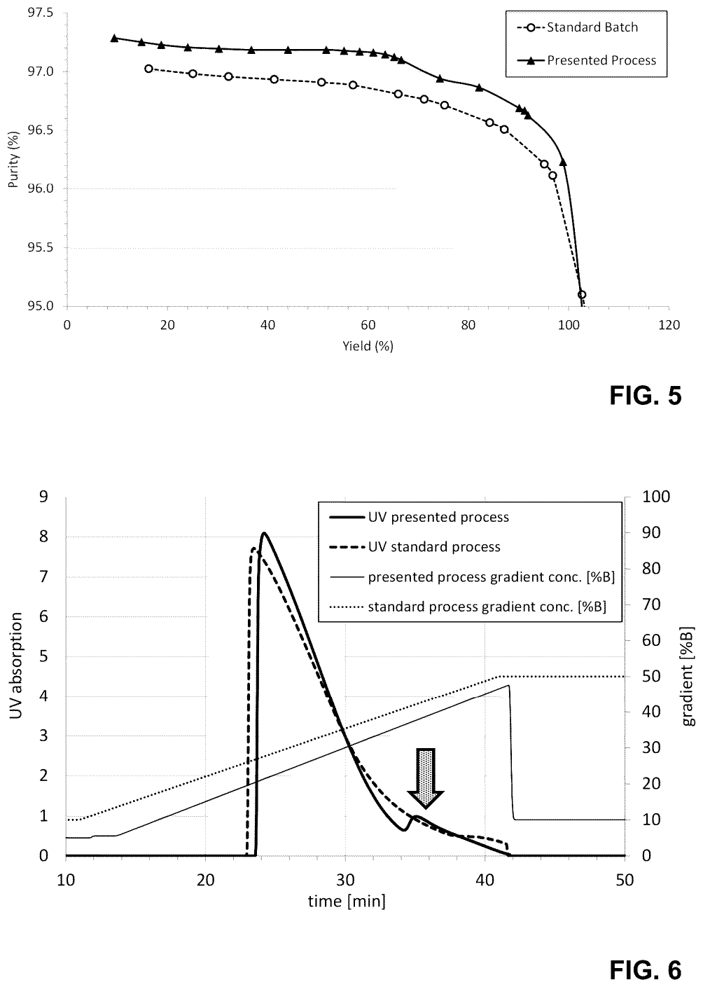

The purity-yield curves are shown in FIG. 5. It is evident that the purity-yield curve of the presented process is superior to the curve of the regular batch chromatography process.

A process performance comparison was carried out for a target purity of 97.0% and is summarized in Table 2. At this purity specification, the product yield is only 16.3% for the standard batch process while it is 66.6% for the presented process, which represents an increase of more than 300%.

The absolute solvent consumption of the presented process was higher than the one of the standard process due to the inline adjustment. However, when considering relative solvent consumption with respect to product produced in specification, the solvent consumption of the presented process is more than 70% lower than the one of the standard process. Given the same load and run duration, the increased yield leads to an analog increase in productivity of around 300%.

TABLE 2
Performance of Example 1 processes

| Performance | Standard | Presented | Absolute | % |
|---|---|---|---|---|
| Parameter | Chromatography | Process | Change | Change |
| Load [g/L] | 10 | 10 | — | — |
| Yield [%] | 16.3 | 66.6 | +50.3 | +309.8% |
| Time [min] | 188 | 188 | — | — |
| S.C. [mL] | 162.5 | 195 | +32.5 | +20% |
| S.C. [L/g] | 20.0 | 5.8 | −14.2 | −71.0% |
| Productivity | 0.52 | 2.13 | +1.6 | +309.8% |
| [g/L/h] | | | | |

Apart from showing superior performance for a given target purity as shown above, it is also clear from FIG. 5. that the presented process enables reaching higher purity values that are inaccessible to standard chromatography: The highest purity value obtained with the standard process is 97.0% with the standard process, while it is 97.3% with the presented process.

Example 2: Simulation of Angiotensin II purification: Mechanistic modelling was carried out to confirm the advantages of the presented process for the purification of Angiotensin II from Solid Phase Peptide Synthesis using reverse phase (RP) chromatography. The adsorption model was based on a Bi-Langmuir isotherm while mass balance and mass transfer were described using a lumped kinetic model. The model was calibrated by peak-fitting using a set of gradient experiments with varying gradient slopes and loads. For the model calibration experiments, YMC Triart Prep C18-S/10 μm 120 A columns with 5 mm inner diameter and 150 mm bed height were used. The feed was 2.0 g/L Angiotensin II in aqueous solution with 5% Acetonitrile and 0.1% trifluoroacetic acid (TFA). The feed purity was 90.0%.

After calibration, the model was used to predict the chromatographic profiles and process performance using a computer simulation with the operating parameters shown in Table 3. Perfect mixing was assumed in-between the two columns in the simulations. Note that in case of presented process, the gradient condition reported in Table 3 refers to the gradient in column 2, i.e. given that the ratio of gradient and inline adjustment flow rates is 1:1, the gradient operated in column 1 is running from 20 to 100%.

TABLE 3
Operating parameters of Example 2

| | | Step | Standard batch | | | Presented process | | |
|---|---|---|---|---|---|---|---|---|
| | | duration | mL/min | mL/min | mL/min | mL/min | mL/min | mL/min |
| Procedure | % B | min | (P1) | (P2) | (P3) | (P1) | (P2) | (P3) |
| Feed | — | 20 | 0 | 0 | 2.0 | 0 | 0 | 2.0 |
| Wash | 10 | 10 | 2.0 | 0 | 0 | 1.0 | 1.0 | 0 |
| Gradient | 50 | 30 | 2.0 | 0 | 0 | 1.0 | 1.0 | 0 |
| Strip | 100 | 10 | 2.0 | 0 | 0 | 1.0 | 1.0 | 0 |
| Re-Equil | 10 | 10 | 2.0 | 0 | 0 | 1.0 | 1.0 | 0 |

The standard batch process was simulated with a bed height of 30 cm while the presented process was simulated for 2×15 cm bed height columns, thus resulting in the same overall bed height.

FIG. 6 shows the chromatograms at the outlet of column 2 obtained by means of the simulations. Despite similar retention time and gradient concentrations, the resolution of the peaks in the tail end of the peak is much better in the presented process than in the standard batch process (indicated by an arrow in FIG. 6).

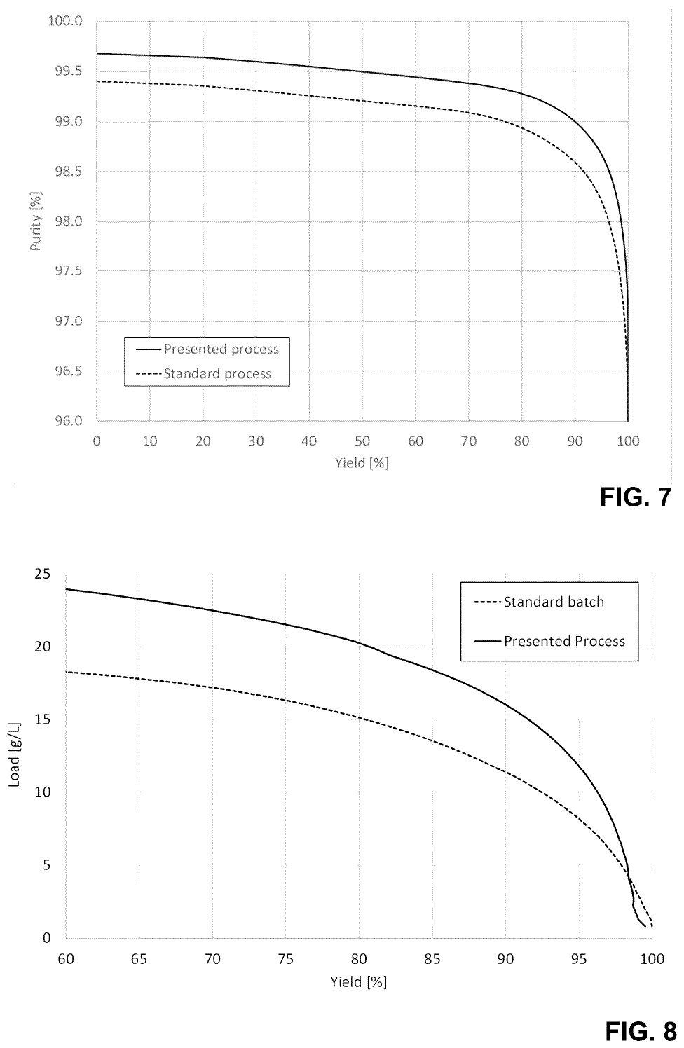

For further evaluation of the two runs, as in example 1, purity-yield curves were generated, based on virtual pools of the product eluate from column 2, starting with the highest purity fractions and increasing the pool size to the sides to include neighboring fractions. Numerically, fractions can be selected much smaller than experimentally, resulting in a smooth curve. The results show a significant improvement of the purity when using the presented process (see FIG. 7).

When plotting the results as load-yield curve for a purity constraint of 99.0%, it becomes clear that the presented process can achieve higher loads for given yields (see FIG. 8). For example, for a target yield of 80%, the Angiotensin II feed can be loaded at 15 g/L (gram Angiotensin II per Liter of packed stationary phase) using the standard single column linear gradient process, while 20 g/L can be loaded with the presented process, thus 33% more. At 90% yield, the corresponding numbers are 11.5 g/L (standard batch) vs 16.2 g/L (presented process), representing a 40% improvement with the presented process.

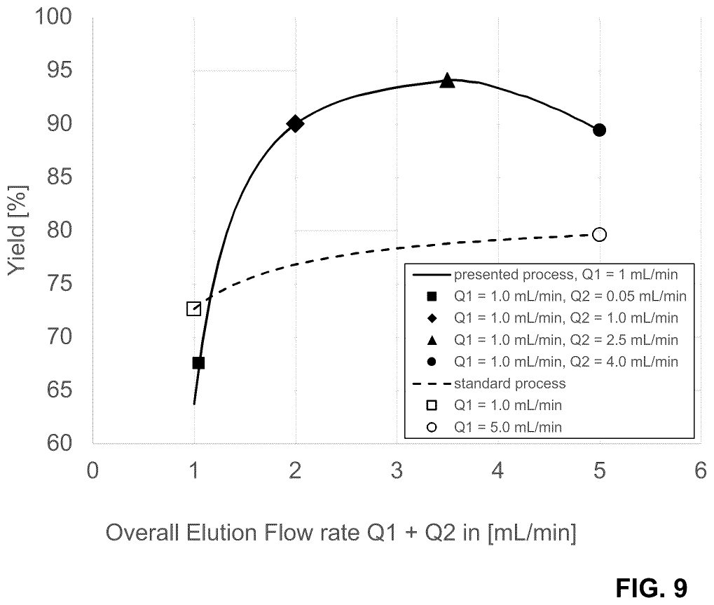

Example 3: Simulation of optimal Inline Adjustment flow rate for Angiotensin II purification: Using the Mechanistic model of example 2, a simulation study was carried out to determine the optimal inline adjustment flow rate for Angiotensin II purification for the selected stationary and mobile phases (see experimental conditions of example 2). For the presented process, the flow rate Q1 of pump P1 was set to Q1=1.0 mL/min, and the flow rate Q2 of pump P2 was varied from Q2=0.2 mL/min to Q2=5 mL/min covering a flow rate ratio range of Q1:Q2 from 1:5 to 5:1. For each simulated experiment, the product yield was determined under a purity constraint of 99.0%. For the standard batch process, the flow rate of Pump P1 was varied between 1.0 mL/min and 5.0 mL/min. The results are reported in FIG. 9. Dedicated simulation points are highlighted and the flow rates Q1 and Q2 are shown in the figure legend. The simulations show an optimum ratio of Q1:Q2 from about 1:2.5 under the chosen conditions for Angiotensin II purification. The maximum can be explained by the following causal relationship: If the ratio of Q1:Q2 is too large, the product is eluted too fast without good resolution of product and impurities. If the ratio of Q1:Q2 is too small, the product is not completely eluted within the gradient.

LIST OF REFERENCE SIGNS

| | |
|---|---|
| P | desired product |
| P1 | pump 1 |
| P2 | pump 2 |
| P3 | pump 3 |
| Q1 | flow rate of pump P1 |
| Q2 | flow rate of Pump P2 |
| IA | Inline adjustment |
| F | feed mixture containing W, P and S |
| W | impurities more weakly adsorbing than the desired product |
| S | impurities more strongly adsorbing than the desired product |
| S.C. | solvent consumption |

1. A chromatographic purification method for the isolation of a desired product from a feed mixture consisting of the desired product and at least two further components, at least one component with impurities more weakly adsorbing than the desired product and at least one component with impurities more strongly adsorbing than the desired product,
said method using two or more chromatographic columns,
wherein a first upstream column has a first column inlet and a first column outlet, and a second downstream column has a second column inlet and a second column outlet,
wherein in one step b) the first upstream column is loaded with feed via the first column inlet,
followed by at least one interconnected step c) in which the columns are interconnected in series for passing eluate containing desired product via the first column outlet to the second column inlet,
wherein in said at least one interconnected step c) the first upstream column is fed with eluent with a gradient in the form of a temporally changing modifier concentration, and
wherein in said at least one interconnected step c) the stream exiting the first column outlet is adjusted in line with inline adjustment eluent before entering the second column inlet at least during a period of gradient elution, and
wherein the inline adjustment eluent is as the eluent fed at the first column inlet but controlled to have a higher or lower modifier concentration than the eluent exiting at the first column outlet, and wherein the modifier difference of the inline adjustment eluent is chosen such that the adherence of the desired product to the stationary phase of the second column (2) is higher than without that difference
wherein
either it involves only two columns, and wherein in said at least one interconnected step c) desired product is collected at the second column outlet;
or it involves said first and said second column and at least one further column, fulfilling, downstream of said second column, the equivalent function of said second column, in that the stream exiting the second column is adjusted in line with inline adjustment solvent before entering the further column inlet at least during the period of gradient elution, and wherein the corresponding inline adjustment solvent is the same as the solvent fed at the first column inlet but controlled to have a different modifier concentration than the solvent exiting at the second column outlet, and wherein the modifier difference of the inline adjustment solvent is chosen such that the adherence of the desired product to the stationary phase of the further column is higher than without that difference, and wherein in said at least one interconnected step c) desired product (P) is collected at the further column outlet.

2. The method according to claim 1, wherein in said at least one interconnected step c) the first column is fed with solvent with a linear gradient, a segment-wise linear gradient, a combination of step and linear gradients, or a combination thereof.

3. The method according to claim 1, wherein the ratio of the flow rate of solvent with gradient in said at least one interconnected step c) at the first column inlet to the flow rate of the inline adjustment is between 5:1 and 1:5.

4. The method according to claim 1, wherein a detector is positioned at the outlet of the second column that is used to monitor the chromatogram, and wherein collection of the desired product is performed at the outlet of the second column controlled by said detector signal.

5. The method according to claim 1, wherein a detector is positioned at the outlet of the first column upstream of the point of inline adjustment for controlling inline adjustment and/or desired product collection.

6. The method according to claim 1, wherein in said at least one interconnected step c) during the transfer of eluate containing the desired product from the first column outlet to the second column inlet the stream entering the second column is mixed inline after or at the point of inline adjustment, by means of a dynamic mixer, a static mixer or a piece of piping with a different diameter than used at the first column outlet and the second column inlet.

7. The method according to claim 1, wherein the columns are disconnected and operated as single columns during at least one part of the procedure that is different from said at least one interconnected step c).

8. The method according to claim 1, wherein it includes the following steps:
a) equilibration of the columns, either in interconnected mode or each column separately, followed by
b) feed, either in interconnected mode or only with the first column disconnected from the second column, in that said feed mixture is introduced via the first column inlet, followed by
c) said at least one interconnected step, followed by
d) regeneration, either in interconnected mode or each column separately, by using conditions cleaning the columns,
wherein the sequence of steps a)-d) is repeated as often as desired and necessary.

9. The method according to claim 1, wherein a diverter valve is used to provide the stream required for inline adjustment in between the columns.

10. The method according to claim 1, wherein the first column and the second column have the same or different bed volumes
and/or wherein the two columns are monoliths or contain membranes
and/or wherein the two columns contain different stationary phases.

11. The method according to claim 1, wherein the modifier is selected from the group consisting of an organic or inorganic solvent or mixture thereof different from a base solvent or mixture thereof of the eluent, an electrolyte in such an organic or inorganic solvent, or a mixture thereof.

12. The method according to claim 1, wherein the two columns, the point of inline adjustment and the mixer are integrated in a single separation device such that they form compartments of a larger column with suited spatial separation and a liquid inlet in between the two compartments.

13. The method according to claim 1, wherein in said at least one interconnected step c) the first upstream column is fed with eluent consisting of a base solvent in mixture with a modifier with a gradient in the form of a temporally changing modifier concentration, wherein the stream exiting the first column outlet is adjusted in line with inline adjustment eluent before entering the second column inlet at least during a period of gradient elution, and wherein the adjustment eluent consists of the base solvent without modifier.

14. The method according to claim 1, wherein the inline adjustment is run as a flow-rate gradient.

15. The method according to claim 1 for the purification of biomolecules, of natural or synthetic origin as well as combinations and modifications as well as fragments thereof.

16. The method according to claim 1, wherein in said at least one interconnected step c) the first column is fed with solvent with a continuous linear gradient.

17. The method according to claim 16, wherein in said at least one interconnected step c) the first column is fed with solvent with a continuous linear gradient during the whole of said interconnected step c).

18. The method according to claim 1, wherein the columns are disconnected and operated as single columns during at least one part of the procedure that is different from said at least one interconnected step c), namely in at least one of the steps of a) equilibration, b) feed and/or wash, d) regeneration.

19. The method according to claim 1, wherein it includes the following steps:
a) equilibration of the columns, either in interconnected mode or each column separately, followed by
b) feed, either in interconnected mode or only with the first column disconnected from the second column, in that said feed mixture is introduced via the first column inlet, followed by washing by introducing solvent without feed mixture by the first column inlet, followed by
c) said at least one interconnected step, followed by
d) regeneration, either in interconnected mode or each column separately, by using conditions cleaning the columns,
wherein the sequence of steps a)-d) is repeated at least 10 times, or at least 100 times.

20. The method according to claim 1, wherein between steps d) and a) in such repetition the position of the columns is interchanged, either after each sequence, or after each 5th or 10^{th} repetition.

21. The method according to claim 11, wherein the modifier is selected from a dissolved salt or a pH, or a combination thereof, wherein
and/or wherein said base solvent is water or a mixture of water with at least one organic solvent or water in a mixture with one or more salts and/or organic solvents one or both in a minor proportion compared with water,
and/or wherein said modifier is an organic solvent or a mixture of water with at least one organic solvent having a higher concentration of said at least one organic solvent than in the base solvent, water or a mixture of water with at least one organic solvent with a different salt or H+ concentration than the base solvent.

22. The method according to claim 15 for the purification of biomolecules, of natural or synthetic origin, selected from the group consisting of nucleic acid molecules, including DNA and RNA molecules, peptides, proteins, including antibodies, carbohydrates, lipids as well as combinations and modifications as well as fragments thereof.
